(19) United States
(12) Patent Application Publication
(10) Pub. No.: US 2013/0005783 A1
(43) Pub. Date: Jan. 3, 2013

(54) Prevention And Treatment Of Diseases Caused By Elevated Levels Of Deoxy-Sphingolipids

(76) Inventor: Thorsten Hornemann, Zurich (CH)

(21) Appl. No.: 13/594,153

(22) Filed: Aug. 24, 2012

Related U.S. Application Data
(63) Continuation of application No. PCT/EP2011/052732, filed on Feb. 24, 2011.

(30) Foreign Application Priority Data
Feb. 24, 2010 (EP) 10154475.7

(51) Int. Cl. A61K 31/198; A61P 25/00; A61P 3/00; C12Q 1/02; A61P 25/18; A61P 11/06; A61K 31/42; A61P 3/10; A61P 25/28

(52) U.S. Cl. 514/380; 514/561; 435/29

(57) ABSTRACT

Substances and methods of use of substances capable of inhibiting serine-palmitoyltransferase (SPT) and/or capable of competing with L-alanine and glycine, including in the reaction catalysed by SPT, including L-serine and D-serine and other compounds, to suppress cytotoxic sphingolipid metabolites, in particular deoxy-sphingolipids. The substances and methods can be used to prevent and treat disease caused by or associated with elevated levels of deoxy-sphingolipids, namely, diabetes (type 1 and type 2 diabetes), particularly diabetic neuropathy, neurodegenerative diseases such as hereditary and sensory neuropathy type I (HSAN1), amyotrophic lateral sclerosis (ALS), Alzheimer disease, other neurological disorders (e.g. depressive disorders, schizophrenia), medication-induced neuriopathies (e.g. induced by treatment with cytostatics like paclitaxel, cis-platin compounds etc.) and other metabolic disorders such as glycogen storage disease type 1a and asthma.

### CROSS-REFERENCE TO RELATED APPLICATIONS

The present application is a continuation of pending International Patent Application PCT/EP2011/052732 filed on Feb. 24, 2011 which designates the United States and claims priority from European Patent Application 10154475.7 filed on Feb. 24, 2012, the content of which is incorporated herein by reference.

### FIELD OF THE INVENTION

The present invention relates to novel means for prevention and treatment of diseases which are caused by or associated with elevated levels of deoxy-sphingolipids, in particular diabetes (type 1 and type 2 diabetes), particularly diabetic neuropathy, neurodegenerative diseases such as hereditary and sensory neuropathy type I (HSAN1), amyotrophic lateral sclerosis (ALS), Alzheimer disease, other neurological disorders (e.g. depressive disorders, schizophrenia), medication-induced neuriopathies (e.g. induced by treatment with cytostatics like paclitaxel, cis-platin compounds etc.) and other metabolic disorders such as glycogen storage disease type 1a and asthma. Specifically, the invention relates to a pharmaceutical suppression of cytotoxic sphingolipid metabolites, in particular deoxy-sphingolipids to prevent and treat these diseases.

### BACKGROUND OF THE INVENTION

Sphingolipids comprise a family of membrane lipids, such as sphingomyelin and glycosphingolipids and bioactive lipids, such as ceramides, sphingosines and dihydro-sphingosines (sphinganines). The first step of the cellular sphingolipid biosynthesis is normally the condensation of serine and palmitoyl-CoA, catalyzed by the serine-palmitoyltransferase (SPT). Although the enzyme shows generally the highest activity with the substrates palmitoyl-CoA and serine, SPT does not strictly depend on these two substrates. The enzyme is also able to metabolize other amino acid substrates, in particular alanine, glycine. This gives rise to the formation of atypical sphingoid base metabolites which lack the C₁ hydroxy group and are therefore classified as deoxy-sphingoid bases. The use of alanine and glycine forms the atypical sphingolipids deoxy-sphinganine (DoxSA, m18:0) and deoxymethyl-sphinganine (DoxmethSA, m17:0), respectively. These metabolites are subsequently N-acetylated by the ceramide synthase (CerS) and finally modified by the ceramide desaturase (DES) forming deoxy-ceramide. Due to the missing C₁ hydroxyl group the deoxy-ceramides are metabolic end products. They can neither be metabolized to complex sphingolipids nor degraded by the classical catabolic pathway which requires the formation of sphingosine-1P as an intermediate metabolite. Consequently, they were shown to accumulate in cells and certain tissues, especially peripheral neurons. Recently, it was found by the inventors that several neurodegenerative diseases are associated with elevated levels of certain sphingolipid metabolites. Especially in the inherited sensory and autonomic neuropathy type 1 (HSN1 or HSAN1) pathologically elevated levels of deoxy-sphingolipids are causally linked to the neurological disorders. Also diabetes and in particular diabetic neuropathy is associated with increased deoxy-sphingolipid levels. Pathological deoxy-sphingolipid levels might also be responsible for other neurodegenerative diseases such as amyotrophic lateral sclerosis (ALS), Alzheimer disease or neurological disorders such as schizophrenia or depression. Deoxy-sphingolipid levels are also elevated during the treatment with certain anti-tumor drugs (e.g. taxol, cis-platin, etoposide) and might thereby contribute to drug-induced neuropathies—a frequent adverse reaction in patients treated with such compounds. Elevated deoxy-sphingolipids are also observed in other metabolic disorders like glycogen storage disease type 1a and might therefore be involved in the pathology of clinical sequelae. Deoxy-sphingolipids are cytotoxic probably by interfering with the filament dynamics of the cytoskeleton and thereby preventing the correct formation of axonal structures in neurons. There is currently no specific treatment for the suppression of deoxy-sphingolipids available.

The technical problem underlying the present invention is the provision of means for preventing or treating diseases caused by or associated with elevated levels of deoxy-sphingolipids.

### SUMMARY OF THE INVENTION

The solution to the above technical problem is provided by the embodiments of the present invention as characterized in the claims.

In particular, the present invention provides a method for the prevention and treatment of diseases caused by or associated with elevated levels of deoxy-sphingolipids, comprising administration to a patient in need thereof a therapeutically effective amount of a deoxy-sphingolipid blocker. Furthermore, the invention relates to the use of such a deoxy-sphingolipid blocker for such prevention and treatment, and to the use of a deoxy-sphingolipid blocker for the manufacture of a medicament for the prevention and treatment of said diseases.

A “deoxy-sphingolipid blocker” according to the present invention is a compound or substance or a composition of such compounds or substances, respectively, capable of inhibiting SPT or capable of competing with the natural reactants leading to deoxy-sphingolipids in the SPT pathway such as L-alanine and glycine. Thus, with regard to the prevention or treatment of diseases caused by or associated with elevated levels of deoxy-sphingolipids, either one compound (substance) capable of inhibiting SPT or a composition of such compounds (substances) may be used for the inventive application. Alternatively, it is possible to use one or more competitors of L-Ala or Gly in the reaction catalysed by SPT. Furthermore, and especially preferred, a combination of one or more of each of SPT inhibitors and competitors of L-Ala/Gly can be used in the prevention and/or treatment of diseases caused by or associated with elevated deoxy-sphingolipids.

The present invention is also directed to a pharmaceutical composition comprising at least one first substance capable of competing with L-alanine and glycine in the reaction catalysed by SPT and at least one second substance capable of inhibiting serine-palmitoyltransferase (SPT), optionally in combination with one or more pharmaceutically acceptable carrier(s), excipient(s) and/or diluent(s).

Furthermore, the present invention is useful in food applications. Thus, there is provided a food additive, a dietary supplement and an animal feed comprising at least one first substance capable of competing with L-alanine and glycine in the reaction catalysed by SPT and at least one second substance capable of inhibiting serine-palmitoyltransferase (SPT).

The preferred compound or substance for competing with D-Ala or Gly in the reaction catalysed by SPT is L-Ser. Preferred compounds or substances inhibiting SPT are D-serine, D-threonine, O-methyl-D,L-serine, sphingofungin B, myriocin, lipoxamycin, viridiofungin A, cycloserine, D-alanine and β-chloroalanine.

The invention further relates to a method of screening for a compound effective in the prevention and treatment of diseases caused by or associated with elevated levels of deoxy-sphingolipids. The invention further relates to compounds selected by these methods of screening.

Specifically, the screening method according to the invention comprises the steps of:
(a) incubating a first population of mammalian cells in the presence of a compound to be tested for its effectiveness in blocking the synthesis of deoxy-sphingolipids and a second population of mammalian cells in the absence of said compound for the same time period;
(b) determining the amount of deoxy-sphingolipids in the first population and in the second population; and
(c) determining whether the amount of deoxy-sphingolipids in the second population is larger than in the first population.

### BRIEF DESCRIPTION OF THE DRAWINGS

FIG. 1 shows the chemical structures of several sphingoid and deoxy-sphingoid bases; A, sphinganine; B, sphingosin; C, deoxy-sphinganine (DoxSA); D, deoxy-sphingosine (DoxSO); E, deoxymethyl sphinganine (DoxmethSA); F, 1-deoxymethyl sphingosine (DoxmethSO).

FIG. 2 shows the chemical structures of several preferred SPT inhibitors; A, sphingofungin B; B, myriocin; C, lipoxamycin; D, viridiofungin A; E, cycloserine, F, β-chloroalanine.

FIG. 3 shows results of studies demonstrating that significantly elevated levels (in pmol/ml plasma; E) of deoxy-sphinganine (DoxSA; left panel) and deoxy-sphingosine (DoxSO, right panel) are found in plasma of patients with diabetes type II (B) and HSAN1 patients which are carriers of the C133Y (C) and C133W (D) mutation in comparison to healthy controls (A).

FIG. 4 shows results of studies demonstrating that significantly elevated levels (in pmol/ml plasma; J) of deoxy-sphinganine (A) and deoxy-sphingosine (B) are found in plasma of patients with Glycogen Storage Disease type Ia (D) but not in controls (C) or patients with Glycogen Storage Disease type Ib (E), type II (F); type III (G); type VI (H) and type IX (I).

FIG. 5 shows a graphical representation of sphinganine (H), deoxy-sphinganine (I) and deoxymethyl-sphinganine (J) levels (in pmol/million cells; K) in SPTLC1-C133W expressing Hek293 cells cultured in the presence of Fumonisin B1 (24 h). Cells were cultivated in normal media without any supplementation (A) or in the presence of 10 mM L-alanine (B), 10 mM glycine (C); 1 mM L-cycloserine (D); 10 mM D-alanine (E), 10 mM D-serine (F) or 10 mM L-serine (G).

FIG. 6 shows the accumulation of sphingnanine (A) and DoxSA (B) in Hek cells cultured in the presence of Fumonsin B1 and increasing concentrations of paclitaxel (I), etoposide (II) and thalidomide (III) (all concentrations in μM; C, pmol/10e6 cells/24 h).

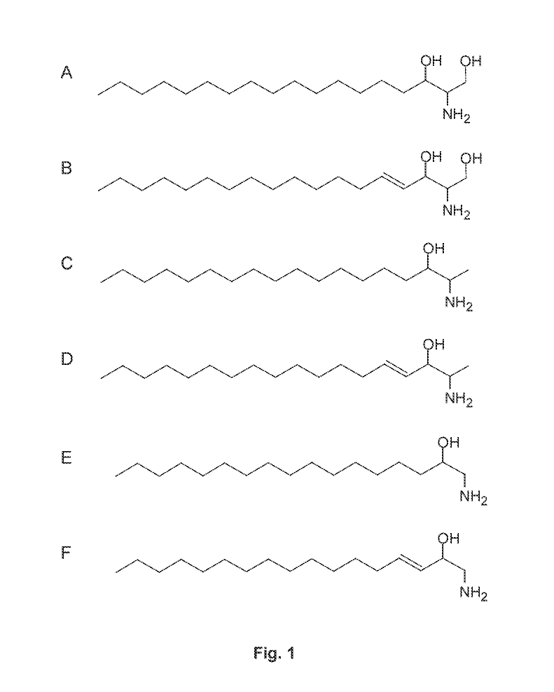

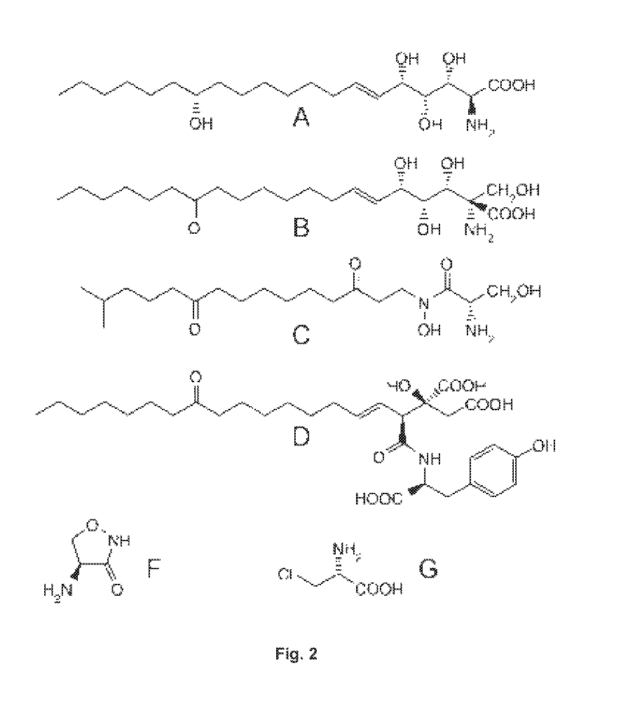

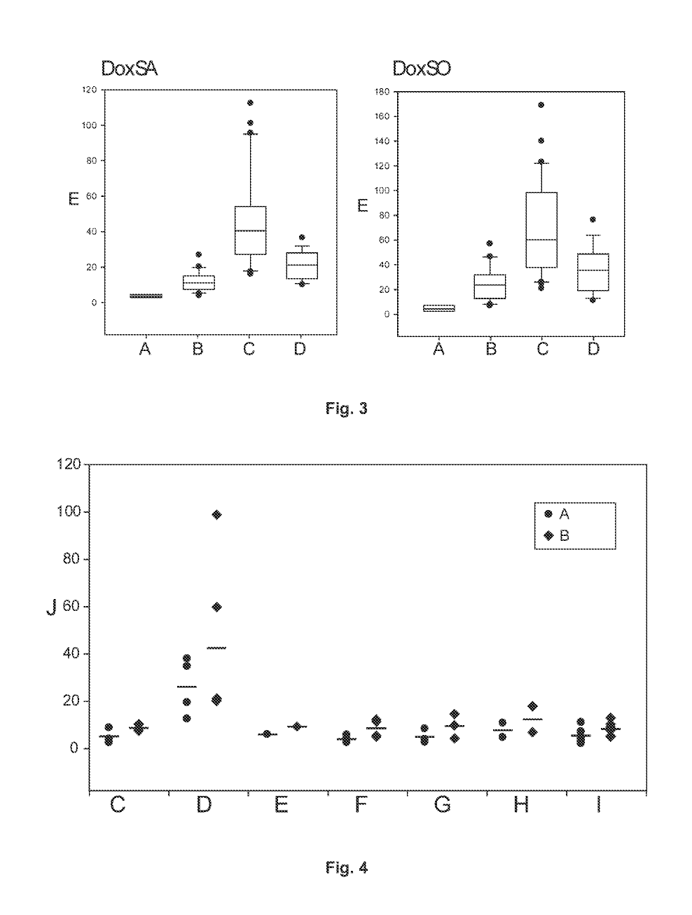

FIG. 7 Panel I shows the suppression of DoxSA production in Hek293 cells at increasing L-serine medium concentrations (B) in the background of varying L-alanine concentrations (C) (A, pmol/10e6 cells/h; B, L-Serine (mM); C, L-alanine (mM)).
Panel II shows the inhibition of DoxSA production in SPTLC1 wt and HSAN1 mutant transfected Hek293 cells at increasing L-serine medium concentrations (B). DoxSA generation by all HSAN1 mutants is efficiently inhibited by small increases in L-erine medium concentration (L-alanine backround of 2 mM) (A, pmol/10e6 cells/h; B,L-Serine mM; C, wildtype; D, mutant C133W; E, mutant C133Y; F, mutant V144D))

FIG. 8 shows graphical representations of the changes in plasma levels of sphingosine (FIG. 8-I), sphinganine (FIG. 8-I), deoxy-sphingosine (FIG. 8-III) and deoxy-sphinganine (FIG. 8-IV) in L-serine (solid line) and L-alanine (dashed line) treated HSAN1 mice. Mice received an L-serine or L-alanine enriched diet (10% L-Serine or L-Alanine w/w) for a period 15 days. The L-Ser-treated mice showed a remarkable reduction in plasma deoxy-sphinganine and deoxy-sphingosine levels already 3-4 days after the start of the treatment as compared to untreated mice. Plasma deoxy-sphingolipid levels remained low during the whole treatment period (A, pmol/ml; B, days).

FIG. 9 shows graphical representations of the changes in deoxy-sphinganine (DoxSA; upper panel) and deoxy-sphingosine (DoxSO; lower panel) plasma levels in HSAN1 patients (C133Y carriers) which received an oral L-Serine treatment over a period of ten weeks. The deoxy-sphingolipid levels remained suppressed until the end of the treatment period (C) and increased again during the washout phase (D). (A, weeks, B, μM; C, treatment phase; D, washout phase).

### DETAILED DESCRIPTION OF THE INVENTION

The present invention provides a method for the prevention and treatment of diseases which are caused by or associated with elevated levels of deoxy-sphingolipids, comprising administering to a patient in need thereof a therapeutically effective amount of a deoxy-sphingolipid blocker, and the use of such blockers in said prevention and treatment and in the manufacture of medicaments for said prevention and treatment.

Diseases caused by or associated with elevated levels of deoxy-sphingolipids are in particular diabetes (type 1 and type 2 diabetes), particularly diabetic neuropathy, neurodegenerative diseases such as hereditary and sensory neuropathy type I (HSAN1), amyotrophic lateral sclerosis (ALS), Alzheimer disease, other neurological disorders (e.g. depressive disorders, schizophrenia), drug and medication-induced neuropathies and metabolic disorders such as glycogen storage disease type 1a and asthma.

A blocker of deoxy-sphingolipids according to the invention is a compound leading to a decrease of circulating and/or intracellular levels of deoxy-sphingolipids.

The term “deoxy-sphingolipid” in the context of the present invention comprises deoxy-sphingoid bases such as deoxy-sphinganine, deoxy-sphingosine, deoxymethyl-sphinganine and deoxymethyl-sphingosine as well as their derivatives further downstream in the pathway triggered by the SPT-catalysed reaction, including N-acyl-deoxy-sphingoid bases and deoxy-ceramides.

Blocking can be achieved by prevention of the reaction of undesired substrates such as L-alanine with SPT (e.g. by increasing the level of L-serine, leading to a competition of serine with alanine and other amino acids for the substrate binding site of SPT) leading to a decrease in the generation of deoxysphingolipids, or by inhibiting SPT activity with an specific inhibitor such as myriocin, cyclo-serine, chloroalanine or by an metabolic upregulation of endogenous competitive substrates such as L-serine or by substances which specifically influence the protein structure of SPT to prevent the generation of deoxy-sphingolipids.

Examples of deoxy-sphingolipid blockers according to the invention are disclosed in the following. However, the invention is not restricted to the blockers disclosed therein, but extends to all blockers of deoxy-sphingolipids or molecules that decrease circulating and/or intracellular levels of deoxy-sphingolipids.

Preferred blockers according to the invention are L- and D-serine, D-alanine and analogues thereof. Analogues of serine and alanine are compounds well known to those in the art and e.g. described by Kayoko Kanda et al. (Journal of General Microbiology (1988), 134, 2747-2755), Woese Cr. et al (J Bacteriol. 1958 December 76(6): 578-88) and Yasuda Y. et al (Microbiol. Immunol. 1985; 29(3): 229-41).

More preferred blockers according to the invention are L-serine, D-serine, D-alanine, D-threonine, O-methyl-DL-serine, sphingofungin B, cycloserine, myriocin, β-chloroalanine, lipoxamycin and viridofungin A, and combinations thereof.

Most preferred blockers according to the invention are L-serine, D-serine and D-alanine and combinations thereof.

For administration, the blocker is preferably in the form of a pharmaceutical preparation comprising the blocker in chemically pure form and optionally a pharmaceutically acceptable carrier and optionally adjuvants. The pharmaceutical compositions comprise from approximately 1% to approximately 99.9% active ingredient.

The administration of a deoxy-sphingolipid blocker may be carried out by any method known to those in the art suitable for delivery to the human organism. For example, in certain aspects of the invention, the deoxy-sphingolipid blocker may be administered orally, by intravenous injection or intraarterial injection. In some aspects, administering comprises transdermal, intraperitoneal, subcutaneous, sustained release, controlled release, delayed release, suppository, or sublingual administration of the deoxy-sphingolipid blocker.

For parenteral administration, preference is given to the use of solutions of the blockers, and also suspensions or dispersions, especially isotonic aqueous solutions, dispersions or suspensions which, for example, can be formed shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations, suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration can also include hard capsules made of gelatin, and also soft, sealed capsules made of gelatin and a plasticizer, such as glycerol or sorbitol. The capsules may contain the active ingredient in the form of granules, or dissolved or suspended in suitable liquid excipients, such as in oils.

The dosage of the active ingredient depends on the species, its age, weight, and individual condition, the individual pharmacokinetic data, and the mode of administration. In the case of an individual having a bodyweight of about 70 kg the daily dose administered of the deoxy-sphingolipid blocker is from 1 mg/kg bodyweight to 1000 mg/kg bodyweight, preferably from 100 mg/kg bodyweight to 500 mg/kg bodyweight, more preferred from 200 mg/kg to 400 mg/kg bodyweight administered as a single dose or as several doses. The deoxy-sphingolipid blocker can be used alone or in combination with one or more further deoxy-sphingolipid blocker(s) or in combination with other drugs.

A preferred combination is a combination of L-serine and D-serine, preferably in a mass ratio of L-serine to D-serine of 1:1 or higher (i.e. more L-serine than D-serine), more preferred in a mass ratio of L-serine to D-serine from about 1:1 to about 1000:1, most preferred from about 10:1 to about 100:1, including 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1 and 99:1. Preferably, a ratio is chosen which leads to a decrease of the level of deoxy-sphingolipids without increasing the level of sphingolipids. The combination can be premixed or mixed shortly before application to the patient in need thereof or also a staggered application is possible.

Furthermore, the invention relates to the use of a deoxy-sphingolipid blocker, in particular the use of a pharmaceutical composition comprising one or more deoxy-sphingolipid blocker(s) as defined above for the prevention or treatment of diseases caused by or associated with elevated levels of deoxy-sphingolipids.

The invention likewise relates to the use of one or more deoxy-sphingolipid blocker(s) as defined above for the manufacture of a medicament for the prevention or treatment of diseases caused by or associated with elevated levels of deoxy-sphingolipids.

The medicaments of the present invention are pharmaceutical compositions prepared in a manner known per se, for example by means of conventional mixing, granulating, coating, dissolving or lyophilizing processes. One may provide the deoxy-sphingolipid blocker by itself, optionally included within a delivery vehicle, such as a liposome or nanoparticle.

The “patient” according to the present invention is human or an animal, in particular mammals such as production animals, e.g. cattle, sheep, pig etc.

Compositions according to the invention comprising at least one compound competing with L-Ala and Gly in the reaction catalysed by SPT and at least one SPT inhibitor are also useful as food additives, dietary supplements and animal feeds. Preferred examples of respective substances and ratios within the composition are as outlined above.

The invention further relates to a method of screening for a compound effective in the prevention or treatment of diseases caused by or associated with elevated levels of deoxy-sphingolipids choosing candidate compounds which selectively decrease or suppress the levels of deoxy-sphingolipids. Those in the art will be familiar with a variety of methods for assessing whether a compound decreases or suppresses the levels of deoxy-sphingolipids. The invention further relates to compounds selected by these methods of screening. Two examples for possible screening methods are described in examples 4 and 5 outlined below.

The following non-limiting examples further illustrate the present invention. These data demonstrate inter alia that L-serine and analogues thereof suppress the levels of deoxy-sphingolipids in cells and plasma and are useful for the prevention and treatment of diseases caused by elevated levels of deoxy-sphingolipids.

### Example 1

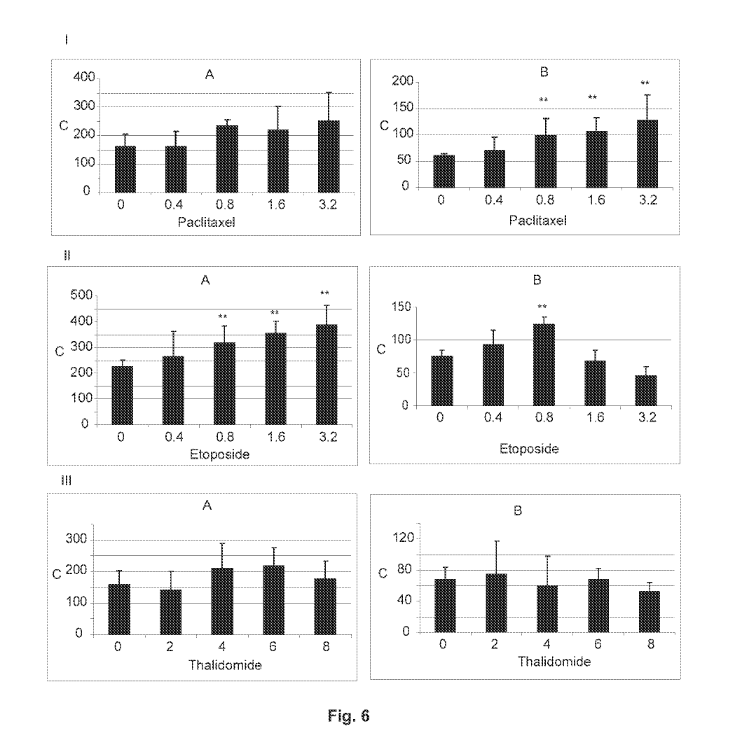

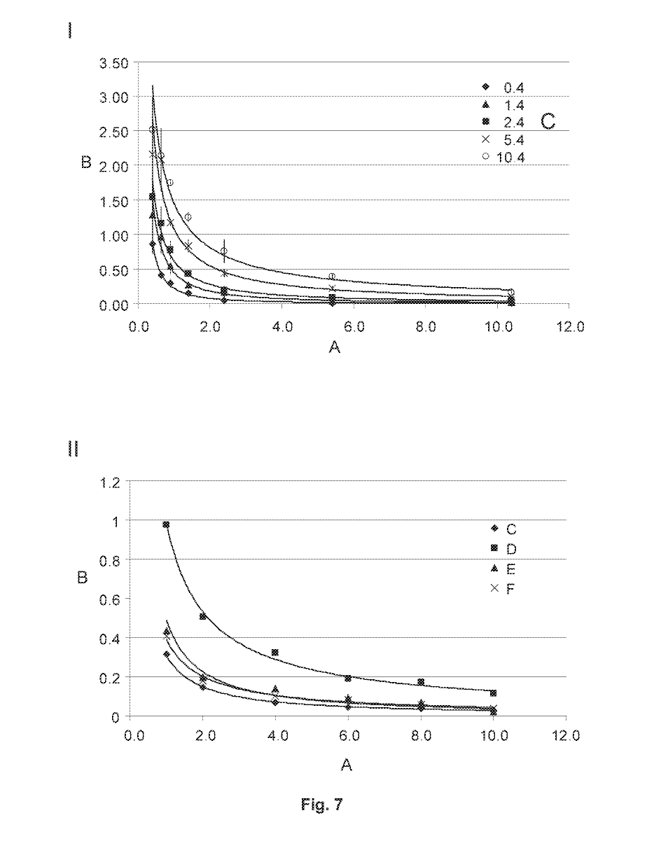

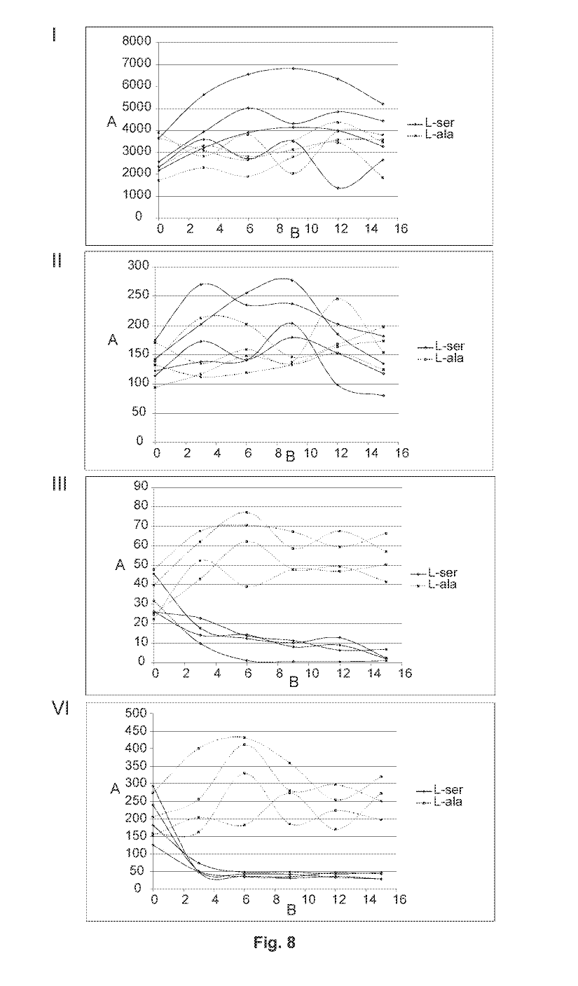

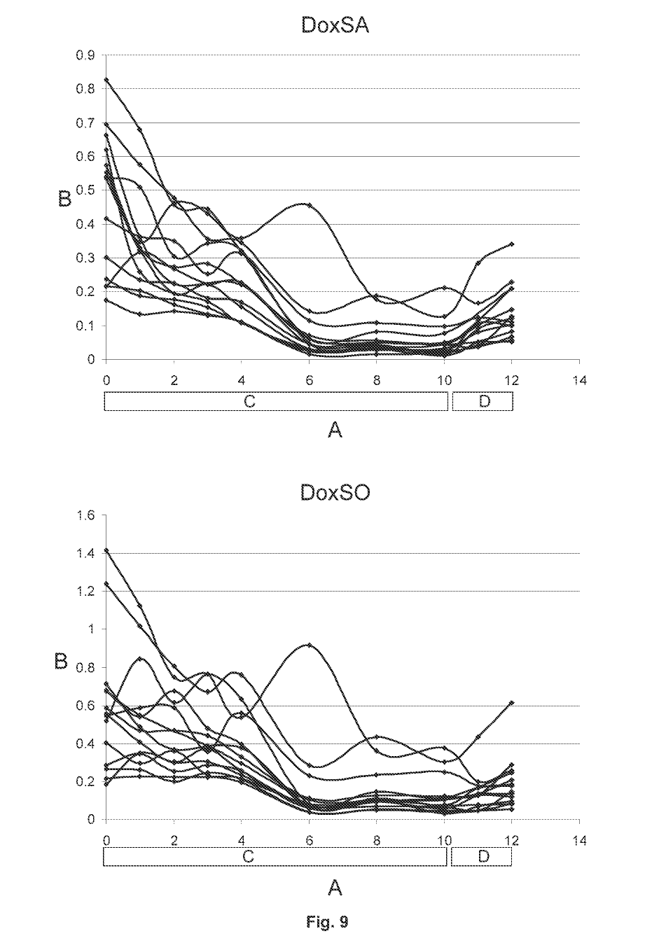

HEK 293 wt cells were cultured in the presence of increasing concentrations of paclitaxel (FIG. 6-I), etoposide (FIG. 6-II) and thalidomide (FIG. 6-III). The cells were additionally treated with fumonisin B1 (FB1), an inhibitor of ceramide synthase (CerS). The inhibition of CerS leads to an intracellular accumulation of the SPT products, namely sphinganine, deoxy-sphinganine. Since the SPT reaction is the rate limiting step in the de novo synthesis pathway, the amount of accumulated sphingoid bases is a measure of the cellular SPT activity. Paclitaxel stimulated DoxSA generation in a dose dependant manner whereas SA generation was not altered (FIG. 6-I). Etoposide increased sphinganine and DoxSA production. SA accumulation increased in a dose dependant manner whereas DoxSA showed a maximum at a concentration of 0.5 mM. In contrast to paclitaxel and etiposide, the non-cytostatic thalidomide (FIG. 6-III) had no effect.

### Example 2

HEK293 wt cells (FIG. 7-I) and Hek cells expressing various mutant forms of SPT (wt, C133W, C133Y, V144D) (FIG. 7-II) were cultured at various L-serine:L-alanine ratios in the presence of FB1 (see above Example 1). A significant decrease in DoxSA formation with increasing L-Serine medium concentrations was observed. This was demonstrated for the wt enzyme with varying L-alanine concentrations (FIG. 7-I) or in the presence of the various HSAN1 mutations (FIG. 7-II). In the second approach L-alanine concentrations were held constant at 2 mM.

### Example 3

HEK293 cells expressing a mutated form of SPT (C133W) were cultured in the presence of various amino acids and structurally related compounds. The cells were additionally treated with fumonisin B1 (see Example 1).

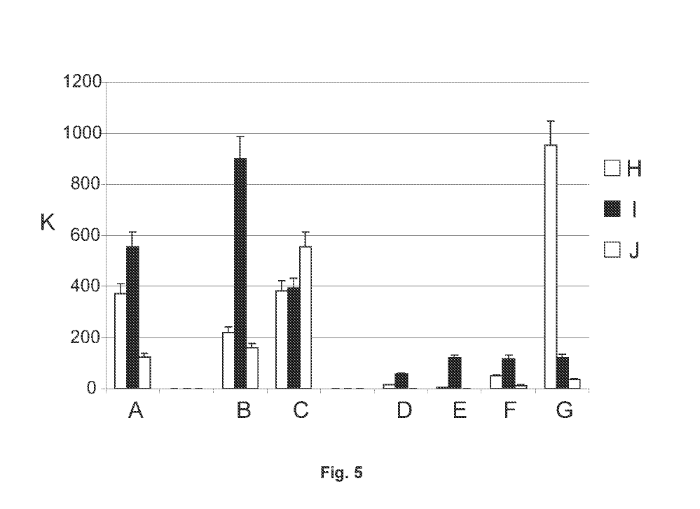

It was observed that the generation of sphingoid base species is selectively influenced by the presence of certain amino acids in the medium. Whereas cycloserine and D-serine act generally inhibitory on SPT activity, the supplementation with L-alanine induces an increased formation of deoxy-sphinganine. Consequently, elevated glycine levels stimulate the formation of deoxymethyl-sphinganine. The supplementation with L-serine, however, suppresses the generation of deoxy-sphingoid bases and on the other hand, stimulates the formation of sphinganine (FIG. 5).

### Example 4

Transgenic mice expressing a mutant (C133W) form of SPTLC1 develop a peripheral neuropathy within 10 to 15 months of life. The development of a neuropathy in these HSAN1 mice correlates with highly elevated deoxy-sphinganine and deoxy-sphingosine plasma levels. The administration of an L-serine enriched diet (10% w/w) to the mice resulted in an up to 80% reduction in plasma deoxy-sphinganine and deoxy-sphingosine levels already after 4-5 days of treatment (FIG. 8). The L-serine-treated mice were protected and did not develop a neuropathy until the end of the study (18 months) whereas untreated control HSAN1 mice displayed severe neuropathic features after 8 to 9 months. In contrast, mice which were fed with an alanine enriched diet showed increased plasma deoxy-sphingolipid levels and developed first neuropathic symptoms already after 2-3 months. These data confirm that the pharmacological suppression of deoxy-sphingolipids provides an effective prevention and treatment for diseases which are caused by pathologically elevated deoxy-sphingolipid levels.

### Example 5

14 HSAN1 patients having the C133Y mutation received an oral serine supplementation for a period of 10 weeks followed by a wash out period of two weeks (FIG. 9). Patients were separated in a low dose and a high dose treatment group (n per group=7) receiving either 5 g or 10 g L-serine/day. The plasma sphingolipid composition was analyzed by an acid/base hydrolysis to release the free sphingoid bases. The treated patients showed a remarkable reduction in plasma deoxy-sphinganine and deoxy-sphingosine levels (FIG. 9) whereby a maximal suppression was reached after 6 weeks of treatment. In both groups an up to 80% reduction in plasma deoxy-sphingolipid levels was observed. No significant differences were seen between the low and the high dose group. During the wash out phase the plasma deoxy-sphingolipid levels raised again. These data show that an oral L-serine supplementation results in an effective suppression of plasma deoxy-sphingolipid levels in humans.

### Example 6

A screening method for compounds which suppresses the generation of deoxy-sphingolipid levels is exemplified as follows: a suitable cell line (e.g. HEK293) is cultured to approximately 80% confluency. Medium is removed, washed with PBS and harvested in 1 ml of PBS by scraping. Cells are pelleted by centrifugation (2500 g, 2 min at 4° C.) and resolved in assay buffer (50 mM HEPES pH 8.0, 0.2% sucrose monolaurate). The protein concentration is adjusted to 2 mg/ml. The final reaction cocktail is composed of 400 μg total protein lysate, 50 mM HEPES (pH 8.0), 0.05 mM palmitoyl-CoA, 20 μM pyridoxal-5′-phosphate, 0.2% sucrose monolaurate and 2 mM L-[U-14C] alanine (0.1 μCi). The assay is performed in the presence (sample) or absence (cntrl) of the putative inhibitory compound X. Final reaction volume is 600 μl. The assay is performed at 37° C. for 60 min. For the negative controls SPT activity is specifically blocked by the addition of myriocin (40 μM). The reaction is stopped by adding 0.5 ml methanolic KOH:CHCl₃ (4:1). Methanolic KOH is prepared by dissolving 0.7 g KOH pellets in 100 ml MeOH. Lipids are extracted under steady agitation for 30 min. Subsequently, 500 μl CHCl₃, 500 μl alkaline water (100 μl NH₃ (2 N) in 100 ml H₂O) and 100 μl NH₃ (2 N) is added in this order. Phases are separated by centrifugation (13000×g, 5 min) and the upper phase discarded. The lower phase is washed three times with alkaline water and the lower organic phase transferred to a scintillation vial. The organic solvent is evaporated under a stream of N₂ and the extracted radioactivity is quantified. The difference in activity between sample and control correlates with the inhibitory potency of compound X.

### Example 7

A further suitable method to screen for putative suppressors of deoxy-sphingolipid synthesis is as follows: a suitable cell line (e.g. HEK293) is cultured to approximately 60% confluency. The compound of interest is added to the culture medium in the appropriate concentration. Additionally, fumonisin B1 (FB1) is added to a final concentration of 10 μg/ml. After 24 h of incubation, cells are washed with PBS and harvested and an aliquot separated for cell counting. The remaining cells are pelleted and extracted in 500 μl extraction buffer (4 volumes MeOH/KOH+1 volume CHCl₃), including an internal reference standard (e.g. deuteriated (d7)-sphinganine, (d7)-sphingosine or synthetic C₁₇-sphingosine). Subsequently, 500 μl chloroform, 500 μl alkaline water and 100 μl 2N ammonia are added, followed by centrifugation (120000×g, 5 min). The upper phase is discarded and the organic phase washed twice with alkaline water. Lipids are dried under a stream of N₂ and resolubilized in 50 μl ethanol (95%) and 100 μl methanol:H₂O (85:15). The lipids are finally derivatized with o-phthaldialdehyde (OPA) and analysed by LC-MS. OPA derivates are separated on a C18 column. Mobile phase is ammonium acetate (5 mM):methanol=17:83 (25 min, isocratic), followed by a wash with 100% methanol (5 min) at a flow rate of 300 μl/min. Deoxysphingoid bases are ionised by APCI (atmospheric pressure chemical ionisation) and analysed in positive ion mode.

1.-11. (canceled)

12. A method for preventing or treating a disease caused by or associated with elevated levels of deoxy-sphingolipids comprising the step of administering to a patient in need thereof a therapeutically effective amount of a substance or combination of substances capable of inhibiting serine-palmitoyltransferase (SPT) and/or capable of competing with L-alanine and glycine in the reaction catalysed by SPT.

13. The method of claim 12 wherein the substance or combination of substances are selected from the group consisting of L-serine, D-serine, D-threonine, O-methyl-D,L-serine, sphingofungin B, myriocin, lipoxamycin, viridiofungin A, cycloserine, D-alanine and β-chloroalanine.

14. The method of claim 12 comprising the administration of at least one first substance capable of competing with L-alanine and glycine in the reaction catalysed by SPT and at least one second substance capable of inhibiting serine-palmitoyltransferase (SPT).

15. The method of claim 14 wherein the first substance is L-serine.

16. The method of claim 14 wherein the second substance is selected from the group consisting of D-serine, D-threonine, O-methyl-D,L-serine, sphingofungin B, myriocin, lipoxamycin, viridiofungin A, cycloserine, D-alanine and β-chloroalanine.

17. The method of claim 14 comprising the administration of L-serine and D-serine.

18. The method of claim 17 wherein L-serine and D-serine are administered in a mass ratio of from 1:1 to 1000:1.

19. The method of claim 18 wherein L-serine and D-serine are administered in a mass ratio of from 10:1 to 100:1.

20. The method claim 17 wherein L-serine and D-serine are administered together in a pharmaceutical composition.

21. The method according to claim 12 wherein the disease is selected from the group consisting of diabetes, diabetic neuropathy, neurodegenerative diseases and metabolic disorders.

22. The method of claim 21 wherein the neurodegenerative disease is selected from the group consisting of hereditary and sensory neuropathy type 1 (HSAN1), amyotrophic lateral sclerosis (ALS), Alzheimer's disease, depressive disorders, schizophrenia and medication-induced neuropathies.

23. The method of claim 21 wherein the metabolic disorder is selected from the group consisting of glycogen storage disease type 1a and asthma.

24. A pharmaceutical composition comprising at least one first substance capable of competing with L-alanine and glycine in the reaction catalysed by SPT and at least one second substance capable of inhibiting serine-palmitoyltransferase (SPT), optionally in combination with one or more pharmaceutically acceptable carrier(s), excipient(s) and/or diluent(s).

25. The pharmaceutical composition of claim 24 wherein the first substance is L-serine.

26. The pharmaceutical composition of claim 24 wherein the second substance is selected from the group consisting of D-serine, D-threonine, O-methyl-D,L-serine, sphingofungin B, myriocin, lipoxamycin, viridiofungin A, cycloserine, D-alanine and β-chloroalanine.

27. The pharmaceutical composition according to claim 24 comprising L-serine and D-serine.

28. The pharmaceutical composition of claim 27 wherein the mass ratio of L-serine to D-serine is from 1:1 to 1000:1.

29. The pharmaceutical composition of claim 28 wherein the mass ratio of L-serine to D-serine is from 10:1 to 100:1.

30. A food additive, dietary supplement or animal feed comprising at least one first substance capable of competing with L-alanine and glycine in the reaction catalysed by SPT and at least one second substance capable of inhibiting serine-palmitoyltransferase (SPT).

31. The food additive, dietary supplement or animal feed of claim 30 wherein the first substance is L-serine.

32. The food additive, dietary supplement or animal feed of claim 31 wherein the second substance is selected from the group consisting of D-serine, D-threonine, O-methyl-D,L-serine, sphingofungin B, myriocin, lipoxamycin, viridiofungin A, cycloserine, D-alanine and β-chloroalanine.

33. The food additive, dietary supplement or animal feed according to claim 30 comprising L-serine and D-serine.

34. The food additive, dietary supplement or animal feed of claim 33 wherein the mass ratio of L-serine to D-serine is from 1:1 to 1000:1.

35. The food additive, dietary supplement or animal feed of claim 34 wherein the mass ratio of L-serine to D-serine is from 10:1 to 100:1.

36. A method of screening for a compound effective in the prevention and/or treatment of diseases caused by or associated with elevated levels of deoxy-sphingolipids comprising the steps of:
incubating a first population of mammalian cells in the presence of a compound to be tested for its effectiveness in blocking the synthesis of deoxy-sphingolipids and a second population of mammalian cells in the absence of said compound for the same time period;
determining the amount of deoxy-sphingolipids in the first population and in the second population; and
determining whether the amount of deoxy-sphingolipids in the second population is larger than in the first population.
